SEQUENCE LISTING (3 sequences)

SEQ ID NO: 1 - protein, 422 aa, Micrococcus candicans ATCC 8456
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 2 - protein, 422 aa, Pseudomonas putida
MNANDNVVIVGTGLAGVEVAFGLRASGWEGNIRLVGDATVIPHHLPPLSKAYLAGKATAESLYLRTPDAYAAQNIQLLGGTQVTAINRDRQQVILSDGRALDYDRLVLATGGRPRPLPVASGAVGKANNFRYLRTLEDAECIRRQLIADNRLVVIGGGYIGLEVAATAIKANMHVTLLDTAARVLERVTAPPVSAFYEHLHREAGVDIRTGTQVCGFEMSTDQQKVTAVLCEDGTRLPADLVIAGIGLIPNCELASAAGLQVDNGIVINEHMQTSDPLIMAVGDCARFHSQLYDRWVRIESVPNALEQARKIAAILCGKVPRDEAAPWFWSDQYEIGLKMVGLSEGYDRIIVRGSLAQPDFSVFYLQGDRVLAVDTVNRPVEFNQSKQIITDRLPVEPNLLGDESVPLKEIIAAAKAELSSA

SEQ ID NO: 3 - protein, 107 aa, Pseudomonas putida
MSKVVYVSHDGTRRELDVADGVSLMQAAVSNGIYDIVGDCGGSASCATCHVYVNEAFTDKVPAANEREIGMLECVTAELKPNSRLCCQIIMTPELDGIVVDVPDRQW

(19) United States
(12) Patent Application Publication
(10) Pub. No.: US 2019/0127321 A1
(43) Pub. Date: May 2, 2019

(54) METHIONINE PRODUCTION

(71) Applicant: EVONIK DEGUSSA GMBH, Essen (DE)

(72) Inventors: Thomas HAAS, Münster (DE); Anja HECKER, Münster (DE); Christian RICHTER, Münster (DE); Thomas BÜLTER, Duisburg (DE)

(73) Assignee: EVONIK DEGUSSA GMBH, Essen (DE)

(21) Appl. No.: 16/095,517

(22) PCT Filed: May 3, 2017
(86) PCT No.: PCT/EP2017/060553

§ 371 Date: Oct. 22, 2018

(30) Foreign Application Priority Data
May 4, 2016 (EP) 16168230.7

(51) Int. Cl. C07C 319/18

(52) U.S. Cl. CPC C07C 319/18

(57) ABSTRACT

There is provided a method of producing a method of producing methionine, the method comprising
contacting vinylglycine or derivatives thereof with at least one free radical methyl mercaptan in a reaction medium.

### FIELD OF THE INVENTION

The present invention relates to a biotechnological method that is capable of producing methionine. In particular, the methionine is formed from at least one vinylglycine.

### BACKGROUND OF THE INVENTION

Amino acids are especially useful as additives in animal feed and as nutritional supplements for human beings. They can also be used in infusion solutions and may function as synthetic intermediates for the manufacture of pharmaceuticals and agricultural chemicals. Compounds such as cysteine, homocysteine, methionine and S-adenosylmethionine are usually industrially produced to be used as food or feed additives and also in pharmaceuticals. In particular, methionine, an essential amino acid, which cannot be synthesized by animals, plays an important role in many body functions. D, L-methionine is presently being produced by chemical synthesis from hydrogen cyanide, acrolein and methyl mercaptan. These petroleum based starting materials such as acrolein are obtained by cracking gasoline or petroleum which is bad for the environment. Also, since the costs for these starting materials will be linked to the price of petroleum, with the expected increase in petroleum prices in the future, prices of methionine will also increase relative to the increase in the petroleum prices.

There are several chemical means of producing methionine. In one example, 3-methylthiopropanal is used as a raw material with hydrocyanic acid in the presence of a base. The reaction results in ammonium carbonate, which is then hydrolysed. In this method, carbon dioxide is introduced into the reaction liquid after hydrolysis, whereby crystallization occurs and methionine is separated as a crystal. Carbon dioxide and hydrogen are used as raw materials for producing methionine using this method. However, a large amount of hydrogen is left over, making this method inefficient.

With the increasing methionine demand, thus microbial production of methionine is always an attractive alternative. Accordingly, there is a need in the art for an alternative biotechnological method of making methionine or a method where most of the steps involved in making methionine are biotechnological.

The pathway for L-methionine synthesis is well known in many microorganisms. E. coli and C. glutamicum are methionine producer strains that have also been described in patent applications WO2005/111202, WO2007/077041, WO2007/012078 and WO2007/135188. Methionine produced by fermentation needs to be purified from the fermentation broth. Cost-efficient purification of methionine relies on producer strains and production processes that minimize the amount of by-products in the fermentation broth. Further, most of these biotechnological methods of producing methionine use nutrients including, but not limited to, carbohydrate sources, e.g., sugars, such as glucose, fructose, or sucrose, hydrolysed starch, nitrogen sources, e.g., ammonia, and sulphur sources e.g., sulphate and/or thiosulfate, together with other necessary or supplemental media components as a starting material. However, this is an expensive raw material and the yield too low to consider this method commercially viable.

Accordingly, there is a need in the art for a more efficient and cost-effective means of producing methionine. In particular, there is a need in the art for a method of producing methionine using biotechnological means or a means where most of the steps are biotechnological and yet cost-efficient.

### DESCRIPTION OF THE INVENTION

The present invention attempts to solve the problems above by providing a method of producing methionine from a substrate that has not been used in production of methionine before. In particular, the present invention provides a method of producing methionine from the substrate, vinylglycine using a chemical means of contacting vinylglycine with a free radical methyl mercaptan. This is advantageous as vinylglycine may be considered an alternative substrate that can be used for the production of methionine, allowing for flexibility of production where there is no reliance on the known substrates that are currently being used for methionine production like acetylhomoserine.

Further, the use of vinylglycine and the free radical methyl mercaptan for methionine production, results in no loss of carbon from the substrate to the desired product, thus making the method efficient. This is because there is no production of a side product like acetic acid that is usually formed when acetylhomoserine is used as the substrate for methionine production. Also, with acetic acid release, the methionine partly absorbs the scent of acetate. The methionine produced using this method thus has a trace of acetate. These problems may be overcome by the method according to any aspect of the present invention. The method according to any aspect of the present invention thus has an advantage of producing L-methionine and/or D-methionine economically through having high conversion rates and short reaction time.

According to one aspect of the present invention, there is provided a method of producing methionine, the method comprising
contacting vinylglycine or derivatives thereof with at least one free radical methyl mercaptan in a reaction medium.

Vinylglyince has been known to be an unstable compound when in aqueous form as at least shown by Friss, Helboe and Larsen in Acta Chem. Scand. (1974): 28B, 317-321. Some non-binding theories for this characteristic of vinylglyince include vinylglyince's overreactivity. Mulzer et al, in journal of Organic Chemistry (1986), 51 (26): 5294-9 also imply that isomerization of the double bond in vinylglycine would have occurred faster than allylglycine, a compound with a similar structure to vinylglycine, resulting in undesired products being formed. The inventors of the present invention were thus surprised by the unexpected stability of vinylglycine in an aqueous medium that reacted with free radical methyl mercaptan to produce the desired product, methionine.

The methionine may be L-methionine and/or D-methionine. In particular, L-methionine is produced. In another example, the methionine may be a methionine peptide.

Vinylglycine has a general chemical formula of C₄H₇NO₂ and a structural formula of:

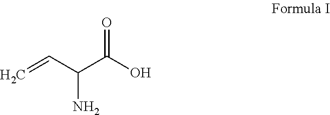

The derivatives of vinylglycine may be selected from the group consisting of rhizobitoxin, aminoethoxyvinylglycine, amine esters of vinylglycine, amide esters of vinylglycine, HCl-Salts of vinylglycine, a protected amino acid of vinylglycine and the like. Protection groups might be Boc, Fmoc, Cbz or ester moieties or a combination of them. In particular, the derivatives of vinylglycine may be selected from the group consisting of rhizobitoxin, aminoethoxyvinylglycine, amine esters of vinylglycine, amide esters of vinylglycine, amides of vinylglycine, esters of vinylglycine and vinylglycine peptides.

The use of vinylglycine as a substrate for methionine production has all the advantages mentioned above and more. For example, the substrate vinylglycine can be synthesized easily from readily available glutamate, the amino acid with one of the highest production volumes in living things. The glutamate may be the L and/or the D isomer.

Vinylglycine and/or derivatives thereof may be formed by any method known in the art. Pellicciari R. et al., in Synthetic Communications (1988), 18 (14): 1715-1731 disclose different ways in which vinylglycine may be produced. In one example, the vinylglycine and/or derivatives thereof may be formed from:

(a) Contacting glutamic acid with a genetically modified cell, wherein the cell comprises
at least a first genetic mutation that increases the expression relative to the wild type cell of an enzyme (E₁) selected from the CYP152 peroxygenase family, and
at least a second genetic mutation that increases the expression relative to the wild type cell of at least one NAD(P)+ oxidoreductase (E₂) and the corresponding mediator protein.

In another example, vinylglycine may also be produced by laminating 3,4-epoxy-1-butene to 4 hydroxy-3-amino-1 butene and then consecutively oxidizing the alcohol group to the acid to form vinylglycine. In another example, the epoxide can be hydrolysed to the diol. The 3,4-dihydroxy-1 butene can be oxidized to the vinylhydroxyacid or the vinylketo acid. These acids can then be reductively aminated to vinylglycine. In one example, vinylglycine may be formed from acrolein exploiting the Strecker or Bucherer reactions or modifications of these reactions. A skilled person would be easily be able to form vinylglycine from these methods known in the art. In another example, multi-step syntheses may be used to act on aminomalonates to form vinylglycine via addition and/or elimination reactions.

In yet another example, vinylglycine and/or derivatives thereof may be produced using a method comprising,

contacting glutamic acid and/or derivatives thereof with an electrolysis medium; and

subjecting the glutamic acid and/or derivatives thereof to anodic electrooxidation in an electrolytic cell to produce the vinylglycine and/or derivatives thereof.

In particular, the electrolytic cell comprises at least two electrodes. In one example, an electric current between the electrodes may have an electric current density about [symbol] 30 mA/cm² of electrode.

Instead of glutamic acid or glutamate, derivatives of glutamate may be used as substrate according to any aspect of the present invention. Derivatives of glutamic acid include esters and/or amides of glutamic acid. In particular, derivatives of glutamic acid may include alkoxy esters, N-Boc protected derivatives, N-Acetyl protected derivatives, salts of glutamic acid, such as sodium glutamate etc., and homo or hetero peptides of glutamic acid.

In one example, a mixture of glutamic acid and at least one derivative of glutamic acid may be used as a substrate according to any aspect of the present invention for producing vinylglycine and/or the respective derivative.

The cell according to any aspect of the present invention may refer to a wide range of microbial cells. In particular, the cell may be a prokaryotic or a lower eukaryotic cell selected from the group consisting of Pseudomonas, Corynebacterium, Bacillus and Escherichia. In one example, the cell may be Escherichia coli. In another example, the cell may be a lower eukaryote, such as a fungus from the group comprising Saccharomyces, Candida, Pichia, Schizosaccharomyces and Yarrowia, particularly, Saccharomyces cerevisiae. The cell may be an isolated cell, in other words a pure culture of a single strain, or may comprise a mixture of at least two strains. Biotechnologically relevant cells are commercially available, for example from the American Type Culture Collection (ATCC) or the German Collection of Microorganisms and Cell Cultures (DSMZ). Particles for keeping and modifying cells are available from the prior art, for example Sambrook/Fritsch/Maniatis (1989).

The phrase “wild type” as used herein in conjunction with a cell or microorganism may denote a cell with a genome make-up that is in a form as seen naturally in the wild. The term may be applicable for both the whole cell and for individual genes. The term ‘wild type’ may thus also include cells which have been genetically modified in other aspects (i.e. with regard to one or more genes) but not in relation to the genes of interest. The term “wild type” therefore does not include such cells or such genes where the gene sequences have been altered at least partially by man using recombinant methods. A wild type cell according to any aspect of the present invention thus refers to a cell that has no genetic mutation with respect to the whole genome and/or a particular gene. Therefore, in one example, a wild type cell with respect to enzyme E₁ may refer to a cell that has the natural/non-altered expression of the enzyme E₁ in the cell. The wild type cell with respect to enzyme E₂, E₃, etc. may be interpreted the same way and may refer to a cell that has the natural/non-altered expression of the enzyme E₂, E₃, etc. respectively in the cell.

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally. The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

The cell and/or enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

Whether or not a nucleic acid molecule, polypeptide, more specifically an enzyme used according to any aspect of the present invention, is recombinant or not does not necessarily have implications for the level of its expression. However, in one example one or more recombinant nucleic acid molecules, polypeptides or enzymes used according to any aspect of the present invention may be overexpressed. The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art. In one example, the cell according to any aspect of the present invention may be genetically modified to comprise at least a first genetic mutation that increases the expression relative to the wild type cell of an enzyme (E₁) selected from the CYP152 peroxygenase family. In this example, the enzyme E₁ may be overexpressed in a wild type cell where the expression of enzyme E₁ may be absent or expressed at the wild type level. Similarly, in the same example or in another example, the enzyme, NAD(P)+ oxidoreductase (E₂) and the corresponding mediator protein may be overexpressed relative to the expression of these enzymes and/or proteins in the wild type cell.

The enzyme (E₁) selected from the CYP152 peroxygenase family used according to any aspect of the present invention may be part of the superfamily of cytochrome P450 enzymes (CYPs) (Malca et al., 2011). Typically, P450 enzymes employ one or more redox partner proteins to transfer two electrons from NAD(P)H to the heme iron reactive center for dioxygen activation, and then insert one atom of Z₂ into their substrates. The enzymes within the family of CYP152 peroxygenases have been identified to exclusively use H₂O₂ as the sole electron and oxygen donors. However, in the cell according to any aspect of the present invention, NAD(P)+ oxidoreductase (E₂) and the corresponding mediator protein may be used as the source of electron and oxygen donors. This is advantageous as in a large scale production of low-cost unsaturated amino acids with a terminal alkenyl group, the use of large amounts of peroxide is cost prohibitive, and high concentration of H₂O₂ can quickly deactivate biocatalysts. Accordingly, the use of NAD(P)+ oxidoreductase (E₂) and the corresponding mediator protein as a source of electrons provides a more cost-effective microbial production of unsaturated amino acids. This may be further explained in Liu et al., 2014.

In particular, enzyme E₁ may be selected from the group consisting of CYP_{SPα} (E₁ₐ), CYP_{BSB} (E_{1b}) (EC 1.11.2.4) and OleT (E_{1c}). More in particular, the enzyme E₁ may be OleT (E_{1c}) or a variant thereof. In one example, enzyme E₁ may comprise the sequence of ADW41779.1. In another example, the enzyme E₁ may have 50, 55, 60, 65, 70, 75, 80, 85, 90, 95, 98, 100% sequence identity to SEQ ID NO: 1.

A skilled person would be capable of identifying the possible sequences of OleT that may be used to carry out the process of forming at least one unsaturated amino acid from at least one amino acid comprising at least two carbonyl groups. In one example, the skilled person may use the disclosure in Liu et al, 2014, Rude M. A, 2011, Schallmey, A., 2011, Fukada H., 1994, Belcher J., 2014 and the like to determine the structure and means of introducing OleT (E_{1c}) into a suitable cell and determining the expression of the enzyme in the cell. OleT (as compared to other H₂O₂-dependent enzymatic reactions) may lead to an artificial electron transfer system to result in higher yield.

The cell used in the method according to any aspect of the present invention may comprise a second genetic mutation that increases the expression relative to the wild type cell of at least one enzyme, the NAD(P)+ oxidoreductase (E₂) and the corresponding mediator protein. These enzymes belong to a family of oxidoreductases that oxidise the mediator protein and accept two electrons. In particular, NAD(P)+ oxidoreductases may use iron-sulphur proteins as electron donors and NAD⁺ or NADP⁺ as electron acceptors. Hannemann et al. discloses a list of various classes of redox-mediators that may be used as enzyme E₂ according to any aspect of the present invention. In one example, artificial/“chemical” redox mediators could transfer electrons either from reductases or electrical sources to the heme iron cluster.

More in particular, the NAD(P)+ oxidoreductase (EC 1.18.1.5) and the corresponding protein may be selected from the group consisting of:

(a) ferredoxin reductase (E₂ₐ) and ferredoxin; or

(b) putidaredoxin reductase (E_{2b}) and putidaredoxin (Schallmey, A., 2011).

In particular, E₂ may be CamA and the mediator protein may be CamB. E₂ may comprise 50, 55, 60, 65, 70, 75, 80, 85, 90, 95, 98, 100% sequence identity to SEQ ID: NO: 2 and/or the mediator protein may comprise 50, 55, 60, 65, 70, 75, 80, 85, 90, 95, 98, 100% sequence identity to SEQ ID: NO: 3.

In one example, in the cell according to any aspect of the present invention E₂ may be ferredoxin reductase (E₂ₐ) where ferredoxin may also be present and E₂ₐ may be capable of functionally interacting with E₁. In particular, the source of E₁ and E₂ may be the same or different. In one example, both E₁ and E₂ may come from the same source, for example from Alcanivorax borkumensis SK2 (accession number YP_691921). In this example, E₂ₐ and ferredoxin may have accession numbers YP_691923 and YP_691920, respectively.

In another example, in the cell used in the method according to any aspect of the present invention E₂ may be putidaredoxin reductase (E_{2b}) where putidaredoxin may also be present and E_{2b} may be capable of functionally interacting with E₁. In one example, E_{2b} may be from the P450_{cam} enzyme system from Pseudomonas putida. For putidaredoxin reductase, typically the amount of enzyme employed may be about 100 to 10,000 ca, 1000 to 5000 ca, 2000 to 4000 ca or in particular 3000 ca. The ca is the unit of activity of putidaredoxin reductase in mediating the oxidation of NADH by ferricyanide and is defined as 1 μmole of NADH oxidised per mg reductase per minute.

E₂ be a recombinant protein or a naturally occurring protein which has been purified or isolated. The E₂ may have been mutated to improve its performance such as to optimise the speed at which it carries out the electron transfer or its substrate specificity. The amount of reductase employed will depend on the exact nature of what is measured and the particular details of the assay but typically, the reductase will be present at a concentration of from 0 to 1000 μM, 0.001 to 100 μM, 0.01 to 50 μM, 0.1 to 25 μM, and in particular from 1 to 10 μM.

The cell used according to any aspect of the present invention may further comprise at least a third genetic mutation that increases the expression relative to the wild type cell of at least one enzyme E₃ capable of cofactor regeneration. In particular, E₃ may be an enzyme capable of NAD(P)H regeneration. More in particular, E₃ may be a dehydrogenase/oxidoreductase which uses NAD(P) as electron acceptor (EC 1.1.1.X). Even more in particular, E₃ may be any enzyme with KEGG no. EC 1.1.1.X in the Brenda database as of 24^{th} Feb. 2014. For example, E₃ may be selected from the group consisting of alcohol dehydrogenase, glycerol phosphate dehydrogenase, histidinol dehydrogenase, shikimate dehydrogenase, lactate dehydrogenase, 3-hydroxyaryl-CoA dehydrogenase, malate dehydrogenase, isocitrate dehydrogenase, glucose-6-phosphate dehydrogenase, formate dehydrogenase, horse liver alcohol dehydrogenase, glucose dehydrogenase, amino acid dehydrogenase, sorbitol dehydrogenase, 20-β-hydroxysteroid dehydrogenase and formaldehyde dehydrogenase. In particular, enzyme (E₃) may be selected from the group consisting of glucose dehydrogenase (E₃ₐ) (EC 1.1.99.10), phosphite dehydrogenase (E_{3b}) (EC 1.20.1.1) and formate dehydrogenase (E_{3c}) (EC 1.2.1.43) where glucose, phosphite and formate are used as reducing agents respectively. The presence of enzyme (E₃) in the cell used in the method according to any aspect of the present invention allows for cofactor regeneration that enables the process of producing unsaturated amino acids from amino acids with two carbonyl groups to be self-sustaining. No external energy would thus have to be introduced into the system of producing unsaturated amino acids. Accordingly, the cell according to any aspect of the present invention may be able to generate at least one unsaturated amino acid from an amino acid with at least two carbonyl groups in the presence of at least enzymes E_{1,} E₂ and/or E₃ without any external energy source needed.

In one example, the glucose dehydrogenase (E₃ₐ) may be NADP+-specific glucose dehydrogenase. The organism that serves as the source of glucose dehydrogenase (E₃ₐ) may not be subject to limitation, and may be a microorganism such as bacteria, fungi, and yeast. For example, a microorganism of the genus Bacillus, in particular Bacillus megaterium, may be the source. In another example, the source may be a microorganism belonging to the genus Cryptococcus, the genus Gluconobacter, or the genus Saccharomyces. In particular, a microorganism belonging to the genus Cryptococcus may be selected, more in particular, the microorganism may be selected from the group consisting of Cryptococcus albi dus, Cryptococcus humicolus, Cryptococus terreus, and Cryptococcus uniguttulatus.

In another example, enzyme E₃ may be phosphite dehydrogenase (E_{3b}) or formate dehydrogenase (E_{3c}). The organism that serves as the source of phosphite dehydrogenase (E_{3b}) or formate dehydrogenase (E_{3c}) may not be subject to limitation, and may be a microorganism such as bacteria, fungi, and yeast.

In one example, the cell according to any aspect of the present invention has increased expression relative to a wild type cell of enzymes E_{1c}, E₂ₐ and E₃ₐ. In another example, the cell according to any aspect of the present invention has increased expression relative to a wild type cell of E_{1c}, E₂ₐ and E_{3b}; E_{1c}, E₂ₐ and E_{3c}; E_{1c}, E_{2b} and E₃ₐ ; E_{1c}, E_{2b} and E_{3b}; or E_{1c}, E_{2b}and E_{3c}.

The teachings of the present invention may not only be carried out using biological macromolecules having the exact amino acid or nucleic acid sequences referred to in this application explicitly, for example by name or accession number, or implicitly, but also using variants of such sequences. The term “variant”, as used herein, comprises amino acid or nucleic acid sequences, respectively, that are at least 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule may be deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In one example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. The term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease may be capable of hydrolysing peptide bonds in polypeptides. The phrase “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, 2, or 1 order of magnitude of the values displayed by the reference molecule with respect to a specific substrate. Similarly, the term “variant” of a nucleic acid comprises nucleic acids the complementary strand of which hybridises, preferably under stringent conditions, to the reference or wild type nucleic acid. A skilled person would be able to easily determine the enzymes E₁, E₂ and/or E₃ that will be capable of making unsaturated amino acids from amino acids with at least two carbonyl groups according to any aspect of the present invention.

An illustration of the difference in the reaction that takes place in the cell according to any aspect of the present invention in the presence of H₂O₂ and the absence of H₂O₂ (i.e. in the presence of enzyme E₂ and the mediator protein instead) is shown in Scheme 1. In particular, in scheme 1 (A), an enzymatic redox-cascade for decarboxylation of a carboxyl group to terminal-alkenyl groups is shown. The electrons are shown to be transferred from a hydride donor (e.g. glucose, formate or phosphite) via CamAB to OleT that catalyses the oxidative decarboxylation of carboxyl groups at the expense of atmospheric O₂ to terminal alkenyl groups. Side products detected are shown in brackets. In scheme 1 (B), the same reaction in the presence of H₂O₂ is shown.

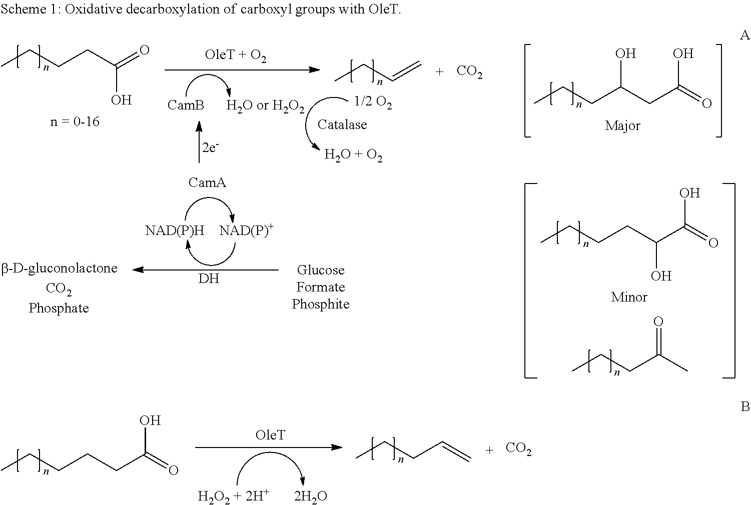

Stringency of hybridisation reactions is readily determinable by one ordinary skilled in the art, and generally is an empirical calculation dependent on probe length, washing temperature and salt concentration. In general, longer probes require higher temperatures for proper annealing, while shorter probes need lower temperatures. Hybridisation generally depends on the ability of denatured DNA to reanneal to complementary strands when present in an environment below their melting temperature. The higher the degree of desired homology between the probe and hybridisable sequence, the higher the relative temperature which may be used. As a result it follows that higher relative temperatures would tend to make the reaction conditions more stringent, while lower temperature less so. For additional details and explanation of stringency of hybridisation reactions, see F. M. Ausubel (1995). The person skilled in the art may follow the instructions given in the manual “The DIG System Users Guide for Filter Hybridization”, Boehringer Mannheim GmbH, Mannheim, Germany, 1993 and in Liebl et al., 1991 on how to identify DNA sequences by means of hybridisation. In one example, stringent conditions are applied for any hybridisation, i.e. hybridisation occurs only if the probe is 70% or more identical to the target sequence. Probes having a lower degree of identity with respect to the target sequence may hybridise, but such hybrids are unstable and will be removed in a washing step under stringent conditions, for example by lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In a particularly preferred embodiment, the temperature is approximately 64° C.-68° C. or approximately 66° C.-68° C. It is possible to adjust the concentration of salt to 0.2×SSC or even 0.1×SSC. Polynucleotide fragments having a degree of identity with respect to the reference or wild type sequence of at least 70, 80, 90, 91, 92, 93, 94, 95, 96, 97, 98, or 99% may be isolated. The term “homologue” of a nucleic acid sequence, as used herein, refers to any nucleic acid sequence that encodes the same amino acid sequence as the reference nucleic acid sequence, in line with the degeneracy of the genetic code.

A skilled person would be capable of easily measuring the activity of each of the enzymes E₁, E₂ and E₃. For example, to determine if the expression of E₂ is increased in a cell, a skilled person may use the assay disclosed in Liu et al, 2014, Rude M.A, 2011, Schallmey, A., 2011, and the like. For example, to determine if the expression of E₂ is increased in a cell, a skilled person may use the assay disclosed in Scheps, D, 2011, Roome et al., Schallmey et al. and the like. The expression of E₃ in a cell, whether it is increased or decreased, may be measured using the assay disclosed at least in Cartel et al. where formate dehydrogenase activity determination (via NAD(P)+ reduction is determined as change in absorbance at 340 nm. A skilled person would easily be able to identify other well-known methods in the art that may be used for measuring the expression of the enzymes used in the cell of the present invention.

Methyl mercaptan also known as methanethiol has a chemical formula of CH₄S and structure of Formula II:

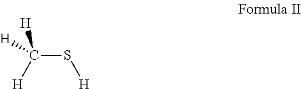

The free-radical addition of a methyl mercaptan to vinylglycine may result in the radicalized methyl mercaptan to acting on the terminal carbon-carbon double bond of vinylglycine to produce 2-amino 4-(methylthio) butanoic acid. The radicalized methyl mercaptan step, also known as Thiol-ene coupling reaction, may also be considered to be relatively selective as no side product may be released when vinylglycine is used as the substrate.

The free radicalization of methyl mercaptan by any means known in the art may result in the breaking of the sulfur-hydrogen bond in methyl mercaptan to produce a methyl mercaptan free radical.

The methyl mercaptan free radical may then act across the terminal carbon-carbon double bond in the vinylglycine. This action may result in the double bond being reduced to a single bond and a methylthio group added according to the Anti-Markovnikov rule at the terminal carbon atom. The unpaired electron on the adjacent, non-terminal carbon atom in the substrate binds with a hydrogen atom supplied by the methyl mercaptan, thereby creating another methyl mercaptan free radical and this continues the addition cycle. The ratio of methyl mercaptan to vinylglycine or derivatives thereof may be 1:1, particularly in the reaction medium. However, a skilled person would be capable of varying this ratio depending on the initiator used to form the radical. In one example, the ratio of methyl mercaptan to vinylglycine or derivatives thereof may be selected from the range of 1:1 to 1:10. In particular, the ratio may be 1.2:1. In one example, the ratio of methyl mercaptan to vinylglycine or derivatives thereof may be selected from 3:1-6:1. This may be advantageous according to any aspect of the present invention as in Thiol-ene coupling reactions, an excess of Thiol may be necessary.

In one example, the free radicalization of methyl mercaptan may be carried out by contacting the methyl mercaptan with at least one free radical initiator. There are several initiators that may be used according to any aspect of the present invention. A skilled person may be capable of identifying these initiators. For example, the free radical initiator may be selected from the group consisting of azobisisobutyronitrile (AIBN), N-bromosuccinimide (NBS), dibenzoyl peroxide (DBPO), Vazo-44 (2,2′-azobis[2-(2-imidazolin-2-yl)propane]dichloride) and the like. When in contact with any of these free radical initiators, the methyl mercaptan may be radicalized to produce a free radical that may then react with the vinylglycine to produce methionine. In one example, AIBN is the free radical initiator. AIBN is thermally stable at room temperature. However, upon being heated to an activation temperature it produces a free radical which may then start the free radical addition chain reaction with vinylglycine. In another example, the Vazo®-44 may be the free radical initiator. The VAZO® series of free radical initiators are available from DuPont Chemicals of Wilmington, Del., U.S.A. In particular, the free radical initiator may be selected from the group consisting of azobisisobutyronitrile (AIBN) and 2,2-Azobis (2-(2-imidazolin-2-yl)propane) dihydrochloride.

In another example, instead of using a chemical agent like a free radical initiator to radicalize methyl mercaptan, an ultraviolet light source may be used. The UV light may be at wavelengths of 300 nm or 365 nm. In particular, the UV light may have a wavelength of 300 nm.

In a further example, free radicalization of the methyl mercaptan may be carried out by a combination of UV light and a photo initiator such as 2,2-Dimethoxy-2-phenylacetophenone (DPAP). In this example, the UV light may have a wavelength of 365 nm.

In one example, free radicalization of the methyl mercaptan may be carried out without an additional initiator. In this example, no chemical initiator and/or UV rays are needed. Radicalization of methyl mercaptan may take place autocatalytically upon heating or may assisted by ultrasonic sound or impurities (e.q. oxygen). A skilled person would be capable of carrying out the radicalization using a variety of means. Reactions without additional chemical initiator may however suffer from low reaction rates and yields.

In all the above examples, the step of free radicalization of methyl mercaptan may be carried out at the same time as the conversion of vinylglycine to methionine. Therefore, both steps of free radicalization and conversion of vinylglycine to methionine may be carried out in the same pot. For example, when a temperature activated free radical initiator such as AIBN is used, the temperature and pressure conditions of the reaction are firstly maintained such that the reactants (i.e. methyl mercaptan, vinylglycine and AIBN) are present as liquids and the temperature is below the activation temperature of the free radical initiator. The order of introduction of the reactants and free radical initiator into the pot is unimportant as the conditions of the reaction mixture in the pot are such that essentially no reaction occurs. When the temperature is increased, the reaction kick starts and radicalized AIBN results in the formation of the free radical of methyl mercaptan which then attacks the C double bond in vinylglycine to form methionine.

In particular, the ratio of free radical initiator to methyl mercaptan may be within the range of 1:10000 to 1:5. More in particular, the ratio of the free radical initiator to methyl mercaptan may be within the range of 1:10000 to 1:10. Even more in particular, the ratio of the free radical initiator to methyl mercaptan may be about 1:1000, 1:500, 1:100, 1:50, 1:20, 1:30, 1:10, 1:3 and the like.

In another example, the pot may have a translucent portion (e.g., a reactor window) where UV light may be shone into the pot. Alternatively, the ultraviolet light source may be disposed within a translucent envelope extending into the pot. The UV light in the reaction pot may then radicalize the methyl mercaptan in the pot. The process may take at least about 5 hours or more. The reaction mixture may then be cooled to room temperature and excess methyl mercaptan may be allowed to volatilize and is removed from the reaction pot. The excess methyl mercaptan may then be recovered for reuse. Methionine may then be left behind in the pot.

In a further example, the pot with a translucent portion may comprise vinylglycine, a photo initiator and methyl mercaptan. The photo initiator may be selected from the group consisting of 2,2-Dimethoxy-2-phenylacetophenone (DPAP), hydroxylcyclohexyl phenyl ketone (HCPK), 2-benzyl-2-N,N-dimethylamino-1-(4-morpholinophenyl)-1-butanone (DBMP), 1-hydroxyl cyclohexyl phenyl ketone, and beozophenone, 2-methyl-1-(4-methylthio)phenyl-2-morpholino propan-1-onc (MMP). In particular, the photo initiator may be DPAP. More in particular, the method according to any aspect of the present invention may comprise free radical methyl mercaptan which may be formed by contacting methyl mercaptan with at least one photo initiator and UV light at a wavelength of 365 nm. Even more in particular, the photo initiator may be DPAP.

Without UV light, no reaction takes place in the pot. When UV light at 365 nm is introduced into the pot by any means known in the art, the photo initiator may be activated to radicalize methyl mercaptan. The free radical of methyl mercaptan may then act on vinylglycine to produce methionine. The excess vinylglycine may then be removed as described above and recycled. The resultant product in the pot may then be only methionine.

The term “contacting”, as used herein, means bringing about direct contact between the glutamic acid used as a substrate, and the cell according to any aspect of the present invention in an aqueous solution. For example, the cell and the glutamic acid may be in different compartments separated by a barrier such as an inorganic membrane. If the glutamic acid is soluble and may be taken up by the cell or can diffuse across biological membranes, it may simply be added to the cell according to any aspect of the present invention in an aqueous solution. In case it is insufficiently soluble, it may be dissolved in a suitable organic solvent prior to addition to the aqueous solution. The person skilled in the art is able to prepare aqueous solutions of amino acids having insufficient solubility by adding suitable organic and/or polar solvents. Such solvents may be provided in the form of an organic phase comprising liquid organic solvent. In one example, the organic solvent or phase may be considered liquid when liquid at 25° C. and standard atmospheric pressure. In another example, the compounds and catalysts may be contacted in vitro, i.e. in a more or less enriched or even purified state, or may be contacted in situ, i.e. they are made as part of the metabolism of the cell and subsequently react inside the cell.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep the cells used in the method according to any aspect of the present invention, for example LB medium in the case of E. coli. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium.

According to any aspect of the present invention, the glutamic acid may be added to an aqueous solution comprising the cell according to any aspect of the present invention. This step may not only comprise temporarily contacting the glutamic acid with the solution, but in fact incubating the glutamic acid in the presence of the cell sufficiently long to allow for an oxidation reaction and possible further downstream reactions to occur, for example for at least 1, 2, 4, 5, 10 or 20 hours. The temperature chosen must be such that the cells according to any aspect of the present invention remains catalytically competent and/or metabolically active, for example 10 to 42° C., in particular 30 to 40° C., more in particular, 32 to 38° C. in case the cell is an E. coli cell.

In particular, the cofactor of the method according to any aspect of the present invention may be NAD+/NADH. More in particular, the method further comprises a coupled process of cofactor regeneration for regenerating the consumed cofactor NAD(P)+. The coupled cofactor regenerating process also comprises the regeneration of the consumed sacrificial glucose, formate, phosphine or the like.

A skilled person would be easily be able to vary the conditions (i.e. pH, pressure, temperature, reaction boosters etc.) to optimize the method according to any aspect of the present invention to produce the highest methionine yield.

In one example, the method according to any aspect of the present invention may be carried out under high pressure conditions. High pressure conditions refer to pressure conditions higher than atmospheric pressure (800-1100 mbar). In the example, high pressure conditions may refer to pressure within the reaction medium according to any aspect of the present invention to be above about 1 bar. In particular, the pressure of the reaction medium according to any aspect of the present invention may be about 1.5, 2, 2.5, 3, 3.5, 4, 4.5, 5, 5.5, 6, 6.5, 7, 7.5, 8, 8.5, 9, 9.5, 10 bar and the like. More in particular, the pressure of the reaction medium may be 2-10, 2-9, 2-8, 2-7, 2-6, 2-5, 3-10, 3-9, 3-8, 3-7, 3-6, 3-5 bar and the like. Even more in particular, the method according to any aspect of the present invention may be carried out in a container (e.g. autoclave, gas cylinder and the like) that may be equipped with a manometer for better control of the pressure.

In another example, the pH in the method according to any aspect of the present invention may be maintained at about 5. In particular, the pH of the reaction mixture may be about 5, 4.5, 4, 3.5, 3, 2.5, 2, 1.5 and the like. More in particular, the pH of the reaction mixture may be selected from 2-5, 2-4, 2-3, 2.5-5, 2.5-4, and 2.5-3.

In a further example, the method according to any aspect of the present invention may be carried out under high pressure and low pH conditions. In particular, the method according to any aspect of the present invention may be carried out at a pressure of 3-10 bar and at a pH of 2-5. More in particular, the method according to any aspect of the present invention may be carried out at a pressure of 3-5 bar and at a pH of 2-3.

In one example, the method according to any aspect of the present invention may include the addition of at least one radical starter also known as a radical initator. The radical initiator may be ammonium peroxydisulfate. A skilled person may be able to select the appropriate radical initiator and use the radical initiator at appropriate concentrations to produce the best yield. The method of producing methionine according to any aspect of the present invention may be a two pot process. In one pot, pot 1, step (a) may be carried out where the cell according to any aspect of the present invention contacts an aqueous medium comprising glutamic acid. The conditions in pot 1 are maintained to optimize production of vinylglycine. A skilled person would be capable of identifying the suitable conditions for optimized activity of the cells in this pot to produce vinylglycine. The vinylglycine may then be concentrated or separated by any means known in the art from pot 1. In one example, vinylglycine may be separated from the solution of pot 1 by precipitation or extraction and the resultant vinylglycine transferred into a second pot, pot 2. In another example, all the contents of pot 1 are transferred to pot 2. Pot 1 may constantly be refilled with glutamic acid and the cells recycled to keep the cost low. In another example, vinylglycine formed is allowed to accumulate in pot 1 before vinylglycine is extracted and transferred to pot 2. In this example, pot 2, before the introduction of vinylglycine may already comprise (i) a temperature activated free radical initiator such as AIBN and methyl mercaptan. When vinylglycine may be introduced into pot 2, the temperature and pressure conditions of pot 2 are firstly maintained such that the reactants (i.e. methyl mercaptan, vinylglycine and AIBN) are present as liquids and the temperature is below the activation temperature of the free radical initiator. When the temperature is increased, the reaction kick starts and radicalized AIBN results in the formation of the free radical of methyl mercaptan which then attacks the C double bond in vinylglycine to form methionine in pot 2.

In another example, vinylglycine from pot 1 may be introduced into pot 2 that comprises methyl mercaptan and which may have a translucent portion (e.g., a reactor window) where UV light may be shone into the pot. Alternatively, the ultraviolet light source may be disposed within a translucent envelope extending into the pot. The UV light introduced into pot 2 may then radicalize the methyl mercaptan in the pot. The process may take at least about 5 hours or more. The reaction mixture may then be cooled to room temperature and excess methyl mercaptan may be allowed to volatilize and is removed from the reaction pot. The excess methyl mercaptan may then be recovered for reuse. Methionine may then be left behind in the pot 2.

In a further example, vinylglycine from pot 1 may be introduced into pot 2 that comprises methyl mercaptan, photo initiator like DPAP and a translucent portion. Without UV light, no reaction takes place in the pot. When UV light at 365 nm is introduced into the pot by any means known in the art, the photo initiator may be activated to radicalize methyl mercaptan. The free radical of methyl mercaptan may then act on vinylglycine to produce methionine. The excess vinylglycine may then be removed as described above and recycled. The resultant product in the pot 2 may then be only methionine.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Synthesis of Methionine Starting From Vinylglycine Via Thiol-Ene-Coupling (TEC)

In a flask (250 mL) is equipped with a reflux condenser vinylglycine (1.011 g, 10.00 mmol, 1.00 eq.) is dissolved in Methanol/Water (1/1, 40 mL) and AIBN (0.164 g, 1.00 mmol, 0.10 eq.) is added. Methyl mercaptan (2.887 g, 2.60 mL, 60.00 mmol, 6.00 eq.) is condensed at −30° C. in a second flask acting as a reservoir. The cooling bath is removed and the reservoir connected to the reaction apparatus to pass the methyl mercaptan through the reaction mixture, while the mixture is heated at 60° C. for 6 hours. The reaction is cooled down to ambient temperature and the formed precipitate collected by filtration to obtain the title compound (as a white crystalline solid of methionine). The structural integrity of the product is confirmed by NMR.

### Example 2

### Synthesis of Methionine Starting From Vinylglycine Via Thiol-Ene-Coupling (TEC) Under Ambient Pressure

In a flask (100 mL) equipped with a reflux condenser vinylglycine (1.011 g, 10.00 mmol, 1.00 eq.) is dissolved in methanol/water (1/1, 40 mL) and AIBN (0.082 g, 0.50 mmol, 0.05 eq.) was added. Sodium thiomethoxide (6.205 g, 60.00 mmol, 6.00 eq.) was placed in a second flask and dissolved in distilled water (10 mL). The second flask (50 mL) was equipped with a dropping funnel (25 mL), which contained hydrochloric acid (6 M, 12 ml). The acid was added dropwise to the thiomethoxide solution over a period of 20 minutes to liberate gaseous methylmercaptan, which was passed into the flask with the vinylglycine. The flask with the vinylglycine solution was kept at 60° C. for 12 h. This flask was connected to gas washing bottles, which contained a sodium hydrogen peroxide solution (dist. water (100 mL), H₂O₂ (35%, 40 mL), NaOH (5.21 g)) in order to destroy escaping methylmercaptan. After heating for 12 h, a nitrogen stream was passed through the reaction mixture for 16 h to push all remaining methylmercaptan into the hydrogen peroxide trap. The residual reaction mixture was evaporated and the off-white residue was analyzed by ¹H-NMR. The NMR measurement revealed that 1% of the vinylglycine was converted to methionine.

### Example 3

Synthesis of Methionine Starting From Vinylglycine Via Thiol-Ene-Coupling (TEC) Under Excess Pressure

Vinylglycine (1.011 g, 10.00 mmol, 1.00 eq.) and AIBN (0.082 g, 0.50 mmol, 0.05 eq.) was dissolved in methanol/water (1/1, 40 mL) in a stainless steel autoclave (300 mL). On one side the autoclave was connected to a methylmercaptan gas cylinder via a U-shaped glass tube. The glass tube acted as an intermediate reservoir for methylmercaptan. On the other side the autoclave was connected to gas washing bottles, which contained a sodium hydrogen peroxide solution (dist. water (100 mL), H₂O₂ (35%, 40 mL), NaOH (5.21 g)) in order to destroy escaping methylmercaptan. The whole apparatus was gently flushed with nitrogen for 20 min. Later, the valves of the autoclave were closed and the glass tube was cooled down below −30° C. The gas cylinder was slowly opened to begin condensing of methylmercaptan inside the glass tube. Having condensed a sufficient amount of methylmercaptan (3 mL, 60 mmol, 6 eq.) the gas cylinder was closed again. Next, the autoclave was cooled to below −30° C. and the valve between autoclave and glass tube was opened. The cooling bath of the glass tube was replaced by a water bath to enable condensation of the methylmercaptan inside the autoclave. After complete evaporation of the methylmercaptan inside the glass tube, the autoclave was sealed and the reaction mixture was heated at 60° C. for 18 h (final pressure at 3.5 bar). The autoclave was then cooled down below −30° C. (no excess pressure) and the apparatus was pressurized with nitrogen (ca. 1.2 bar). The valves of the autoclave were carefully opened and a nitrogen stream was passed through the reaction mixture for 22 h to push all remaining methylmercaptan into the hydrogen peroxide trap.

Cooling of the autoclave after heating sometimes caused a low-pressure in the vessel. In such low-pressure, when the autoclave was opened to the peroxide trap and the applied nitrogen pressure on the other side was not high enough, the peroxide solution was sucked in. Therefore, to prevent this, the vessel was frozen, nitrogen pressure applied and the valve opened between autoclave and glass tube and then the valve between autoclave and trap carefully opened. The nitrogen pressure was increased to establish a stable nitrogen stream through the apparatus when the solution started to get sucked in. increase.

Then the autoclave was opened and the yellowish residue was suspended in methanol/water (1/1, 40 mL). The precipitated methionine was filtered off, washed with methanol (2×20 mL) and dried in vacuum. When the condensation of the methylmercaptan proceeded very slowly and the internal pressure of the glass tube raised to 0.6 bar excess pressure, the autoclave valves were opened for some seconds and then later closed. The internal atmosphere was enriched with methylmercaptan and the condensation was faster and proceeded at lower pressures (0.2-0.4 excess pressure).

Fresh peroxide solution was added in the gas washing bottles before starting the final elimination of methylmercaptan.

The methionine (0.50 g, 34%, purity_{(NMR)}: 98%) was obtained as an off-white solid. The combined filtrates were evaporated and the residue (0.90 g) was analyzed by ¹H-NMR. The NMR measurement revealed that the residue is a mixture of vinylglycine and methionine in a ratio of 27 to 73. Therefore, the overall conversion from vinylglycine to methionine can be calculated to 81%.

### Example 4

### Synthesis of Methionine Starting From Vinylglycine Via Thiol-Ene-Coupling (TEC) Under Excess Pressure at Lower pH

Vinylglycine (1.011 g, 10.00 mmol, 1.00 eq.) and AIBN (0.082 g, 0.50 mmol, 0.05 eq.) was dissolved in methanol/water (1/1, 40 mL) in a stainless steel autoclave (300 mL). Acetic acid (1.201 g, 20.00 mmol, 2.00 eq.) was added to the solution (pH=2.5-3). Later, methylmercaptan (3 mL, 60 mmol, 6 eq.) was condensed into the reaction mixture and the autoclave was sealed. The procedure as disclosed in Example 3 was used to handle methylmercaptan. The reaction mixture was heated at 68° C. for 23 h (final pressure at 2.9 bar). The reaction was cooled down and the methylmercaptan was removed. The solvent of the obtained suspension was removed and the residue was washed with EtOH (2×10 mL). The off-white solid (0.77 g) was dried in vacuum and analyzed by ¹H-NMR. The NMR measurement revealed that the residue is a mixture of vinylglycine and methionine in a ratio of 13 to 87.

### Example 5

### Synthesis of Methionine Starting From Vinylglycine Via Thiol-Ene-Coupling (TEC) Under Excess Pressure Without Radical Starter

Vinylglycine (1.011 g, 10.00 mmol, 1.00 eq.) was dissolved in methanol/water (1/1, 40 mL) in a stainless steel autoclave (300 mL). Later, methylmercaptan (3 mL, 60 mmol, 6 eq.) was condensed into the reaction mixture and the autoclave was sealed. The procedure as disclosed in Example 3 was used to handle methylmercaptan. The reaction mixture was heated at 66° C. for 22 h (final pressure at 3.2 bar). The reaction was cooled down and the methylmercaptan was removed. The solvent of the obtained solution was removed and the residue was dried in vacuum. Analysis by ¹H-NMR revealed that the residue (1.06 g) is a mixture of vinylglycine and methionine in a ratio of 88 to 12. (Conversion rate of vinylglycine to methionine: 12%.)

### Example 6

### Synthesis of Methionine Starting From Vinylglycine Via Thiol-Ene-Coupling (TEC) Under Excess Pressure With Peroxo Radical Starter

Vinylglycine (1.011 g, 10.00 mmol, 1.00 eq.) and ammonium peroxodisulfate (0.024 g, 0.10 mmol, 0.01 eq.) was dissolved in methanol/water (1/1, 40 mL) in a stainless steel autoclave (300 mL). Afterwards, methylmercaptan (3 mL, 60 mmol, 6 eq.) was condensed into the reaction mixture and the autoclave was sealed. (For more details how to handle methylmercaptan see procedure above.) The reaction mixture was heated at 66° C. for 23 h (final pressure at 3.2 bar). The reaction was cooled down and the methylmercaptan was removed. The solvent of the obtained solution was removed and the residue was dried in vacuum. Analysis by ¹H-NMR revealed that the residue (1.12 g) is a mixture of vinylglycine and methionine in a ratio of 77 to 23. (Conversion rate of vinylglycine to methionine: 23%.)

1. A method of producing methionine, the method comprising
contacting vinyiglycine or derivatives thereof with at least one free radical methyl mercaptan in a reaction medium.

2. The method according to claim 1, wherein the ratio of methyl mercaptan to vinylglycine or derivatives thereof is 1:1-1:10.

3. The method according to claim 1, wherein the free radical methyl mercaptan is formed by contacting methyl mercaptan with at least one free radical initiator in the reaction medium.

4. The method according to claim 3, wherein the free radical initiator is selected from the group consisting of azobisisobutyronitrile (AIBN), N-bromosuccinimide (NBS), dibenzoyl peroxide (DBPO) and 2,2-Azobis (2-(2-imidazolin-2-yl)propane) dihydrochloride.

5. The method according to claim 3, wherein the ratio of free radical initiator to methyl mercaptan is selected from the range of 1:10000 to 1:10.

6. The method according to claim 3, wherein the free radical initiator is dibenzoyl peroxide (DBPO).

7. The method according to claim 1, wherein the free radical methyl mercaptan is formed by contacting methyl mercaptan with UV light.

8. The method according to claim 7, wherein the UV light has a wavelength of 300 nm.

9. The method according to claim 1, wherein the free radical methyl mercaptan is formed by contacting methyl mercaptan with at least one photoinitiator and UV light at a wavelength of 365 nm.

10. The method according to claim 9, wherein the photoinitiator is selected from the group consisting of hydroxylcyclohexyl phenyl ketone (HCPK), 2-benzyl-2-N, N-dimethylamino-1-(4-morpholino phenyl)-1-butanone (DBMP), 1-hydroxyl cyclohexyl phenyl ketone, and beozophenone, 2-methyl-1-(4-methylthio)phenyl-2-morpholino propan-1-onc (MMP).

11. The method according to claim 1, wherein the vinylglycine or derivatives thereof is formed from:
(a) contacting glutamic acid with a genetically modified cell, wherein the cell comprises
at least a first genetic mutation that increases the expression relative to the wild type cell of an enzyme (E₁) selected from the CYP152 peroxygenase family, and
at least a second genetic mutation that increases the expression relative to the wild type cell of at least one NAD(P)+ oxidoreductase (E₂) and the corresponding mediator protein.

12. The method according to claim 11, wherein
E₁ is selected from the group consisting of CYP_{SPα} (E₁ₐ)CYP_{BSB} (E_{1b}) and OleT (E_{1c}): and
E₂ and the corresponding mediator protein are selected from the group consisting of ferredoxin reductase (E₂ₐ) and ferredoxin; and putidaredoxin reductase (E_{2b}) and putidaredoxin.

13. The method according to claim 11, wherein
E₁ OleT (E_{1c}) and comprises at least 60% sequence identity to SEQ ID NO:1; and/or
E₂ comprises 60% sequence identity to SEQ ID NO:2 and the mediator protein comprises 60% sequence identity to SEQ ID NO:3.

14. The method according to claim 11, wherein the cell further comprises at least a third genetic mutation that increases the expression relative to the wild type cell of at least one enzyme E₃ capable of NAD(P)H regeneration.

15. The method according to claim 14, wherein the enzyme E₃ is selected from the group consisting of glucose dehydrogenase, phosphite dehydrogenase and formate dehydrogenase.
